(19) United States
(12) Patent Application Publication
(10) Pub. No.: US 2019/0162652 A1
(43) Pub. Date: May 30, 2019

(54) Screening Test Paper Reading System

(71) Applicant: Cheng-Hao Ko, Hsinchu County (TW)

(72) Inventor: Cheng-Hao Ko, Hsinchu County (TW)

(21) Appl. No.: 15/885,776

(22) Filed: Jan. 31, 2018

(30) Foreign Application Priority Data
Nov. 30, 2017 (CN) 2017800017911

(51) Int. Cl. G01N 21/01; G01N 33/52; G01N 21/84; G01N 21/78; B01L 9/00

(52) U.S. Cl. CPC G01N 21/01; G01N 33/521; G01N 2021/752; G01N 21/78; B01L 9/52; G01N 21/8483

(57) ABSTRACT

The invention discloses a screening test paper reading system comprising a screening test paper, a test paper carrier, a delivery unit and a reading unit. The screening test paper is provided with a plurality of reaction zones thereon for reacting with a plurality of specific samples, and changing their own color. The test paper carrier is provided with a bearing groove thereon for accommodating the screening test paper and the test paper carrier is provided with a plurality of positioning parts and a gearing part thereon. The delivery unit has a driving device and a detection device. When the detection device detects a plurality of positioning parts on the test paper carrier, the driving device is activated and the test paper carrier is moved by the gearing part so that the reading unit retrieves a color signal on each the reaction zone, whereby the screening test paper reading system can achieve an effect of a fast screening and protection of the screening test paper.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

This invention generally relates to a screening test paper reading system, especially to a screening test paper reading system which can achieve an effect of a fast screening and protection of the screening test paper.

### Description of Related Arts

As people pay more attention to their own physical health, screening including physical screening or environmental screening has become a popular topic. FIG. 1 is a schematic view of a conventional screening test paper. As shown in FIG. 1, the screening test paper 10 is provided with reaction zones 11. When users drop a sample on the reaction zone 11, a reagent of the reaction zone 11 reacts with the sample, and the color of the reaction zone 11 changes. The users can determine if normal or not of the sample according to the color change and color contrast of the reaction zone. However, the accuracy of color change identification is very poor by human's eyes. When a precision screening is performed, it is often the case that spectrum analyzers are used.

However, when using a spectrum analyzer for screening, users have to provide a screening test paper on a corresponding position manually for the spectrum analyzer to retrieve a color signal on each the reaction zone. It may cause the screening test paper wearing and easily lead to a problem that the color signal cannot be retrieved due to the inaccurate placement. The above way is not practical because it wastes a lot of time to adjust and correct the position of the screening test paper.

### SUMMARY OF THE PRESENT INVENTION

It is an object of the invention to provide a screening test paper reading system which can achieve an effect of a fast screening and protection of the screening test paper.

With the above object in view the invention discloses a screening test paper reading system comprising a screening test paper provided with a plurality of reaction zones thereon for reacting with a plurality of specific samples, and changing their own color; a test paper carrier provided with a bearing groove thereon for accommodating the screening test paper and the test paper carrier provided with a plurality of positioning parts and a gearing part thereon; a delivery unit having a driving device and a detection device; and a reading unit, wherein when the detection device detects a plurality of positioning parts on the test paper carrier, the driving device is activated and the test paper carrier is moved by the gearing part so that the reading unit retrieves a color signal on each the reaction zone.

According to the screening test paper reading system, the delivery unit further comprises a caging device for limiting a position of the test paper carrier when it is moved.

According to the screening test paper reading system, the driving device is a DC motor.

According to the screening test paper reading system, the driving device is a stepper motor.

According to the screening test paper reading system, the gearing part is a rack.

According to the screening test paper reading system, the screening test paper is applied to specific samples comprising saliva, urine, blood, excrement, water, food, toxic and hazardous substance or microorganism.

According to the screening test paper reading system, the detection device is an infrared detector, optical detector, laser detector or image sensor.

According to the screening test paper reading system, the reading unit is an optical wavelength spectrometer or spectrum analyzer.

According to the screening test paper reading system, the caging device further comprises an opening with a “C”, “ [symbol] ” or “ [symbol] ” form to limit a position of the test paper carrier when it is moved.

### BRIEF DESCRIPTION OF THE DRAWINGS

FIG. 1 is a schematic view of a conventional screening test paper.

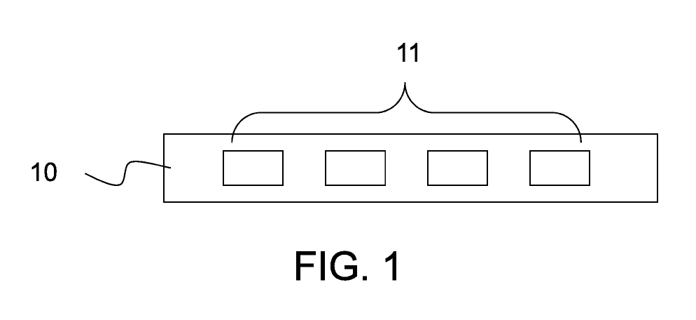

FIG. 2 shows a structural exploded view of a screening test paper reading system of the invention.

FIG. 3 shows a lateral cross-sectional view of a screening test paper reading system of the invention.

### DETAILED DESCRIPTION OF THE PREFERRED EMBODIMENT

Some terms are used to refer to specific components in the specification and subsequent claims. It is to be understood by the those of ordinary skill in the art that the hardware manufacturers may use different terms to describe the same component. The components in the specification and subsequent claims are not distinguished by their names, but by the difference in the function. The term “comprising” throughout the specification and subsequent claims is an open-ended term, so the term “comprising” is to be construed as “comprising, but not limited to”.

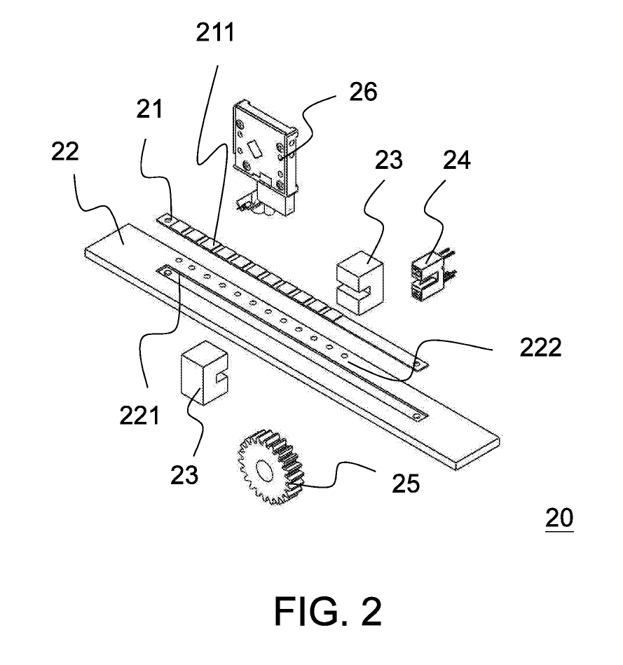

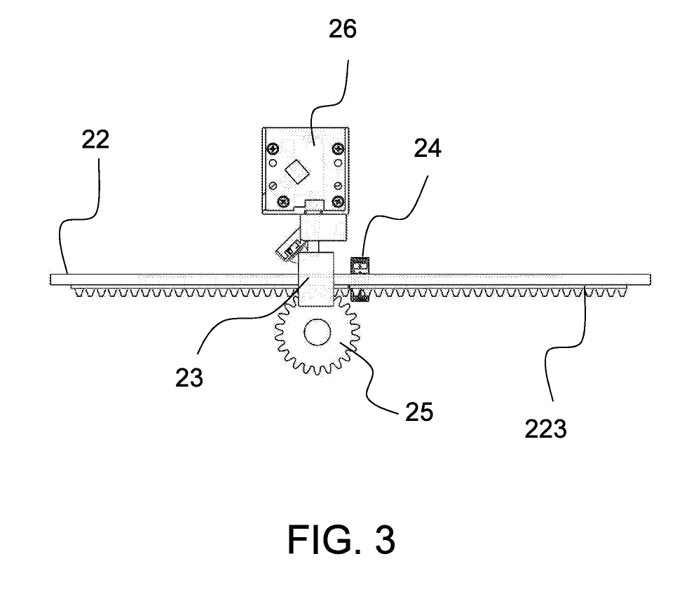

FIG. 2 and FIG. 3 are respectively a structural exploded view and a lateral cross-sectional view of a screening test paper reading system of the invention. As shown in the drawings, the screening test paper reading system 20 of the invention comprises a screening test paper 21, a test paper carrier 22, a delivery unit and a reading unit 26. The screening test paper 21 is provided with a plurality of reaction zones 211 thereon for reacting with a plurality of specific samples (e.g., urine or blood, etc.), and changing their own color. The test paper carrier 22 is provided with a bearing groove 221 thereon for accommodating the screening test paper 21 and the screening test paper 21 is fixed on the test paper carrier 22. The test paper carrier 22 is provided with a plurality of positioning parts 222 (referring to positioning points) and a gearing part 223 (e.g., a rack) thereon. The delivery unit has a driving device 25 (e.g., a DC motor or a stepper motor) and a detection device 24. When the detection device 24 detects a plurality of positioning parts 222 on the test paper carrier 22, the driving device 25 is activated and the test paper carrier 22 may be moved to a corresponding position by gearing part 223 so that the reading unit 26 retrieves a color signal on each the reaction zone 211. In the above embodiment, the specific samples comprise saliva, urine, blood, excrement, water, food, toxic and hazardous substance or microorganism. The detection device 24 may be an infrared detector, optical detector, laser detector or image sensor. The reading unit 26 may be an optical wavelength spectrometer or spectrum analyzer.

In addition, in order to enable the test paper carrier 22 will not be detached from or offset from the driving device 25 when the test paper carrier 22 is moved, the delivery unit is further provided with a caging device 23 to make the test paper carrier 22 limited to a “ [symbol] ” form opening in the caging device 23 as the test paper carrier 22 moving for achieving the above purpose. The “ [symbol] ” form opening of the caging device 23 may be replaced with “C” or “ [symbol] ” opening.

Following the above-mentioned embodiment, as determining a value of urine glucose of a patient, a screening test paper specifically for urine glucose can be directly used to determine the value. The urine to be determined contacts with a reaction zone 211 of the screening test paper 21 in a form of drip or infiltration, and the component in the reaction zone 211 reacts with the urine to be determined and changes the color of the reaction zone 211. In addition, the reaction zone 211 may contain a variety of determining zones to determine glucose, protein, pH value, latent blood value or other physiological values in urine to achieve an effect of multiple values with one screening. After the urine to be determined contacts and reacts with the reaction zone 211 of the screening test paper 21, the screening test paper 21 may be provided in the bearing groove 221 of the test paper carrier 22, and the test paper carrier 22 is inserted into a spectrum analyzer (i.e., the screening test paper reading system 20 of the invention).

When the test paper carrier 22 is inserted to a certain depth, the detection device 24 may detect a plurality of positioning parts 222 on the test paper carrier 22 (shown as dot appearance on the test paper carrier 22 in FIG. 2), at this time the screening test paper reading system 10 of the invention may determine that there is a screening test paper inserted and carrying out screening operations is desired, and the driving device 25 such as a DC motor or a stepper motor is activated and the test paper carrier 22 may be moved to the corresponding position by the gearing part 223 such as a rack. Generally speaking, it is an aspect of sucking up the test paper carrier 22. Because the specifications of the screening test paper 21 and the moving distance of the test paper carrier 22 driven by the driving device 25 are fixed or predetermined, it will be quite accurate for the test paper carrier 22 to move to the corresponding position for the reading unit 26 to retrieve the color signal of each reaction zone 211, which is positioning in one time. Next, the reading unit 26 may retrieve the color signal of each reaction zone 211, and the color signal is treated by a spectrometer to achieve a purpose of fast screening.

Moreover, because the screening test paper 21 is provided in the bearing groove 221 of the test paper carrier 22 that is not the way of directly contacting with the spectrum analyzer, the screening test paper 21 is protected by the test paper carrier 22, and not easy to wear.

It is to be understood by the those of ordinary skill in the art that the above preferable embodiments of the invention are described in detail and the various possible modifications and alterations of the embodiments could be made, without departing from the scope and spirit of the invention. The various embodiments such as caging unit are not limited by the way carrying out the embodiments in the specification.

What is claimed is:

1. A screening test paper reading system comprising:
a screening test paper provided with a plurality of reaction zones thereon for reacting with a plurality of specific samples, and changing their own color;
a test paper carrier provided with a bearing groove thereon for accommodating the screening test paper and the test paper carrier provided with a plurality of positioning parts and a gearing part thereon;
a delivery unit having a driving device and a detection device; and
a reading unit,
wherein when the detection device detects the positioning parts on the test paper carrier, the driving device is activated and the test paper carrier is moved by the gearing part so that the reading unit retrieves a color signal on each the reaction zone.

2. The screening test paper reading system according to claim 1, wherein the delivery unit further comprises a caging device for limiting a position of the test paper carrier when it is moved.

3. The screening test paper reading system according to claim 1, wherein the driving device is a DC motor.

4. The screening test paper reading system according to claim 1, wherein the driving device is a stepper motor.

5. The screening test paper reading system according to claim 1, wherein the gearing part is a rack.

6. The screening test paper reading system according to claim 1, wherein the screening test paper is applied to specific samples comprising saliva, urine, blood, excrement, water, food, toxic and hazardous substance or microorganism.

7. The screening test paper reading system according to claim 1, wherein the detection device is an infrared detector, optical detector, laser detector or image sensor.

8. The screening test paper reading system according to claim 1, wherein the reading unit is an optical wavelength spectrometer or spectrum analyzer.

9. The screening test paper reading system according to claim 2, wherein the caging device further comprises an opening with a “C”, “ [symbol] ” or “ [symbol] ” form to limit a position of the test paper carrier when it is moved.
